(19) United States
(12) Patent Application Publication
(10) Pub. No.: US 2022/0259539 A1
(43) Pub. Date: Aug. 18, 2022

(54) BIOINTERFACES FOR GROWING SEAWEED

(71) Applicant: W. L. Gore & Associates, Inc., Newark, DE (US)

(72) Inventor: Norman E. Clough, Landenberg, PA (US)

(21) Appl. No.: 17/622,687

(22) PCT Filed: Jun. 26, 2020
(86) PCT No.: PCT/US2020/039951

§ 371 Date: Dec. 23, 2021

Related U.S. Application Data
(60) Provisional application No. 62/867,704, filed on Jun. 27, 2019.

(51) Int. Cl. C12M 1/12; A01G 33/00; A01G 18/20; B01D 71/32; B01D 71/44

(52) U.S. Cl. CPC C12M 25/02; A01G 33/00; B01D 71/44; B01D 71/32; A01G 18/20

(57) ABSTRACT

Biointerfaces configured to retain and viably maintain non-mammalian cells are disclosed. The biointerfaces may include one or more of a nutrient phase, an adhesive, a bioactive agent, a liquid containing phase. The biointerfaces may be patterned. The biointerfaces may specifically retain and viably retain specific non mammalian cell types such as spores of seaweed. The biointerfaces are used for growing seaweed such as dulse and kelp.

### CROSS-REFERENCE TO RELATED APPLICATION

This application claims the benefit of Provisional Application No. 62/867,704, filed Jun. 27, 2019, which is incorporated herein by reference in its entirety for all purposes.

### FIELD

The present disclosure relates generally to non-mammalian biointerfaces, and more specifically to biointerfaces configured to retain and viably maintain non-mammalian.

### BACKGROUND

While significant research and development has gone into the development of biointerfaces for mammalian (i.e., human) cells, there is a need for biointerfaces specifically tailored to non-mammalian cells.

For example, the current process to cultivate seaweed from spores involves using textured nylon “culture strings” or “seed strings” to which the spores weakly attach during a lab-based seeding process and are then nourished through external nutrient systems. The culture string containing weakly attached juvenile seaweed (gametophytes and sporophytes) is then wound onto ropes at a seaweed farm, where the ropes are subsequently placed under water. The process is inherently variable in terms of yield and throughput due in large part to the ease in which the seaweed can be damaged from, for example, currents, changes in temperature, and nutrient availability. Further, poor packaging and handling can result in damage and loss of juvenile seaweed. Current approaches to improving stability of juvenile seaweed on culture strings is focused on the surface texture of existing fibers. Indeed, fiber texture of culture strings is very important to the success of seaweed cultivation. However, improvements to surface texture are limited.

### SUMMARY

Various embodiments are directed toward non-mammalian biointerfaces configured to retain and viably maintain non-mammalian cells.

According to one example (“Example 1”), the non-mammalian biointerface comprises a microstructure configured to retain and viably maintain viruses or non-mammalian cells, the microstructure being characterized by an average inter-fibril distance up to and including 200 μm.

According to another example (“Example 2”), the non-mammalian biointerface comprises a microstructure configured to retain and viably maintain viruses or non-mammalian cells, the microstructure configured to retain viruses or non-mammalian cells at least partially within the microstructure, the microstructure being characterized by an average pore size of up to and including 200 μm.

According to another example (“Example 3”) further to Example 1, the microstructure is characterized by an average inter-fibril distance from 1 to 200 μm.

According to another example (“Example 4”) further to any one of preceding Examples 1 or 2, the microstructure is characterized by an average pore size from 1 to 200 μm.

According to another example (“Example 5”) further to any one of preceding Examples 1 to 4, the microstructure is configured to retain spores.

According to another example (“Example 6”) further to any one of preceding Examples 1 to 4, the microstructure is configured to retain bacteria.

According to another example (“Example 7”) further to any one of preceding Examples 1 to 4, the microstructure is configured to retain microbes.

According to another example (“Example 8”) further to any one of preceding Examples 1 to 7, the non-mammalian biointerface comprises a nutrient phase associated with at least a portion of the non-mammalian biointerface.

According to another example (“Example 9”) further to Example 8, at least a portion of the nutrient phase is located within the microstructure, located on the microstructure, or located both within the microstructure and on the microstructure.

According to another example (“Example 10”) further to any one of preceding Examples 8 or 9, the nutrient phase is present as a coating on a surface of the non-mammalian biointerface.

According to another example (“Example 11”) further to any one of preceding Examples 8 to 10, the nutrient phase acts as a chemoattractant to selectively attract the viruses or non-mammalian cells to predetermined locations of the non-mammalian biointerface to which the nutrient phase is applied or included.

According to another example (“Example 12”) further to any one of preceding Examples 8 to 11, the nutrient phase is configured to i) promote growth and/or proliferation of the viruses or non-mammalian cells within the microstructure, and/or ii) maintain and/or encourage attachment to and integration within the microstructure of the viruses or non-mammalian cells to the microstructure.

According to another example (“Example 13”) further to any one of preceding Examples 1 to 12, a liquid containing phase is associated with at least a portion of the non-mammalian biointerface.

According to another example (“Example 14”) further to preceding Example 13, at least a portion of the liquid containing phase is entrained within the microstructure, entrained on the microstructure, or entrained both within the microstructure and on the microstructure.

According to another example (“Example 15”) further to any one of preceding Examples 13 or 14, the liquid containing phase is present as a coating on a surface of the non-mammalian biointerface.

According to another example (“Example 16”) further to any one of preceding Examples 3 to 15, the liquid containing phase comprises a hydrogel, a slurry, a paste, or a combination thereof.

According to another example (“Example 17”) further to any one of preceding Examples 1 to 16, the non-mammalian biointerface includes a plurality of viruses or non-mammalian cells retained by the microstructure of the non-mammalian biointerface.

According to another example (“Example 18”) further to any one of preceding Examples 1 to 17, the non-mammalian biointerface includes a fibrillated material having a microstructure including a plurality of fibrils defining an average inter-fibril distance.

According to another example (“Example 19”) further to any one of preceding Examples 1 to 18, the non-mammalian biointerface comprises a material having an average density from 0.1 to 1.0 g/cm³.

According to another example (“Example 20”) further to Example 19, the non-mammalian biointerface includes a growth medium comprising the material, and a ratio of the average inter-fibril distance (μm) to the average density (g/cm³) of the fibrillated material is from 1 to 2000.

According to another example (“Example 21”) further to any one of preceding Examples 1 to 20, the non-mammalian biointerface is configured as a fiber, a membrane, a woven article, a non-woven article, a braided article, a knit article, a fabric, a particulate dispersion, or combinations of two or more of the foregoing.

According to another example (“Example 22”) further to any one of preceding Examples 1 to 21, the microstructure is provided by a plurality of particles in a dispersion formulated for deposition onto a backer layer or a carrier substrate to form the non-mammalian biointerface.

According to another example (“Example 23”) further to any one of preceding Examples 1 to 21, the non-mammalian biointerface includes at least one of a backer layer, a carrier layer, a laminate of a plurality of layers, a composite material, or combinations thereof.

According to another example (“Example 24”) further to any one of preceding Examples 1 to 23, at least a portion of the non-mammalian biointerface is hydrophilic.

According to another example (“Example 25”) further to any one of preceding Examples 1 to 24, at least a portion of the non-mammalian biointerface is hydrophobic.

According to another example (“Example 26”) further to any one of preceding Examples 1 to 25, one or more portions of the non-mammalian biointerface is hydrophobic and one or more portions of the non-mammalian biointerface is hydrophilic such that the non-mammalian biointerface is configured to selectively encourage retention of the viruses or non-mammalian cells in the one or more hydrophilic portions of the non-mammalian biointerface.

According to another example (“Example 27”) further to any one of preceding Examples 1 to 26, the non-mammalian biointerface includes a bioactive agent associated with the non-mammalian biointerface.

According to another example (“Example 28”) further to any one of preceding Examples 1 to 27, the non-mammalian biointerface includes an adhesive applied to a surface of the microstructure, imbibed within the microstructure of the non-mammalian biointerface, or both applied to a surface of the microstructure and imbibed within the microstructure of the non-mammalian biointerface.

According to another example (“Example 29”) further to any one of preceding Examples 1 to 28, the non-mammalian biointerface includes a salt associated with the microstructure of the non-mammalian biointerface.

According to another example (“Example 30”) further to preceding Example 29, the salt is sodium chloride (NaCl).

According to another example (“Example 31”) further to any one of preceding Examples 1 to 30, the microstructure includes a pattern of higher density portions and lower density portions, the lower density portions corresponding to a portion of the microstructure configured to retain spores on and/or within the microstructure of the microstructure.

According to another example (“Example 32”) further to preceding Example 31, the lower density areas are characterized by a density of 1 g/cm³ or less and the higher density portions are characterized by a density of 1.7 g/cm³ or more.

According to another example (“Example 33”) further to any one of preceding Examples 1 to 32, the microstructure includes a pattern of higher porosity portions and lower porosity portions, the lower porosity portions corresponding to a portion of the microstructure configured to retain viruses or non-mammalian cells within the microstructure of the non-mammalian biointerface.

According to another example (“Example 34”) further to any one of preceding Examples 1 to 32, the microstructure includes a pattern of higher porosity portions and lower porosity portions, the higher porosity portions corresponding to a portion of the microstructure configured to retain viruses or non-mammalian cells within the microstructure of the non-mammalian biointerface.

According to another example (“Example 35”) further to any one of preceding Examples 1 to 34, the microstructure includes a pattern of greater inter-fibril distance portions and lower inter-fibril distance portions, the lower inter-fibril distance portions corresponding to the portion of the microstructure configured to retain spores within the microstructure of the non-mammalian biointerface.

According to another example (“Example 36”) further to any one of preceding Examples 1 to 34, the microstructure includes a pattern of greater inter-fibril distance portions and lower inter-fibril distance portions, the greater inter-fibril distance portions corresponding to the portion of the microstructure configured to retain spores within the microstructure of the non-mammalian biointerface.

According to another example (“Example 37”) further to any one of preceding Examples 31 to 36, the pattern is an organized or selective pattern.

According to another example (“Example 38”) further to any one of preceding Examples 31 to 36, the pattern is a random pattern.

According to another example (“Example 39”) further to any one of preceding Examples 1 to 38, the non-mammalian biointerface comprises an expanded fluoropolymer.

According to another example (“Example 40”) further to any one of preceding Examples 8 to 39, the biointerface comprises an expanded fluoropolymer wherein the nutrient phase is co-blended with the expanded fluoropolymer.

According to another example (“Example 41”) further to Example 39 or 40, the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), and expanded polytetrafluoroethylene (ePTFE).

According to another example (“Example 42”) further to any one of preceding Examples 1-38, the non-mammalian biointerface comprises an expanded thermoplastic polymer.

According to another example (“Example 43”) further to preceding Example 42, the expanded thermoplastic polymer is one of: expanded polyester sulfone (ePES), expanded ultra-high-molecular-weight polyethylene (eUHMWPE), expanded polylactic acid (ePLA), and expanded polyethylene (ePE).

According to another example (“Example 44”) further to any one of preceding Examples 1 to 38, the non-mammalian biointerface comprises an expanded polymer.

According to another example (“Example 45”) further to any one of preceding Examples 8 to 38 and 44 the non-mammalian biointerface comprises an expanded polymer wherein the nutrient phase is co-blended with the expanded polymer.

According to another example (“Example 46”) further to any one of preceding Examples 44 or 45, the expanded polymer is expanded polyurethane (ePU).

According to another example (“Example 47”) further to any one of preceding Examples 1-38, the non-mammalian biointerface comprises a polymer formed by expanded chemical vapor deposition (CVD)

According to another example (“Example 48”) further to Example 47, the polymer formed by expanded CVD is expanded polyparaxylylene (ePPX).

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a scanning electron microscopy (SEM) micrograph depicting a microstructure of a non-mammalian biointerface in accordance with some embodiments.

FIG. 2 is an SEM micrograph depicting the microstructure pictured in FIG. 1, but at a higher magnification.

FIG. 3 is an SEM micrograph depicting a microstructure of a non-mammalian biointerface in accordance with some embodiments.

FIG. 4 is an SEM micrograph depicting the microstructure pictured in FIG. 3, but at a higher magnification.

FIG. 5 is a schematic illustration depicting a microstructure of a non-mammalian biointerface in accordance with some embodiments.

FIG. 6 is the micrograph of FIG. 2 with cartoon representations of non-mammalian cells of either 10 μm or 30 μm overlaid thereon in inter-fibril spaces in accordance with some embodiments.

FIG. 7A is a cross-sectional SEM micrograph depicting ingrowth of dulse seaweed into a microstructure of a non-mammalian biointerface in accordance with some embodiments.

FIG. 7B is a cross-sectional SEM micrograph depicting the ingrowth pictured in FIG. 7A, but at a higher magnification.

FIG. 7C is a cross-sectional optical fluorescence microscopy micrograph depicting ingrowth of dulse seaweed into a microstructure of a non-mammalian biointerface in accordance with some embodiments.

FIG. 8 presents a surface SEM micrograph (top panel) depicting a microstructure of a cultivation substrate prior to seeding with sugar kelp spores in accordance with some embodiments, and an optical fluorescence microscopy micrograph (bottom panel) depicting the cultivation substrate following seeding with sugar kelp spores and germination thereof.

FIG. 9 presents two surface SEM micrographs taken at different magnifications depicting juvenile dulse ingrowth into a microstructure in accordance with some embodiments.

FIG. 10 is a surface optical fluorescence microscopy micrograph depicting ingrowth of dulse seaweed into a microstructure of a non-mammalian biointerface in accordance with some embodiments.

FIG. 11 is an SEM micrograph depicting the superficial surface attachment of developing seaweed to the surface fibers of a high-density material in accordance with some embodiments.

FIG. 12 is an SEM micrograph depicting a woven non-mammalian biointerface in accordance with some embodiments.

FIG. 13 is an SEM micrograph depicting a commercially available porous polyethylene.

FIG. 14 is a collection of photographs depicting growth of dulse on a gel processed polyethylene membrane in accordance with some embodiments (Membrane 1), and a commercially available porous polyethylene (Membrane 2).

FIG. 15 is a collection of photographs depicting growth of kelp on a gel processed polyethylene membrane in accordance with some embodiments (Membrane 1), and a commercially available porous polyethylene (Membrane 2).

FIG. 16 is a photograph depicting growth of dulse on a patterned membrane in accordance with some embodiments.

FIG. 17 photograph depicting growth of kelp on a patterned membrane in accordance with some embodiments.

FIG. 18 is a photograph depicting juvenile sugar kelp sporophyte attachment to a membrane in accordance with some embodiments.

Persons skilled in the art will readily appreciate the accompanying drawing figures referred to herein are not necessarily drawn to scale, but may be exaggerated or represented schematically to illustrate various aspects of the present disclosure, and in that regard, the drawing figures should not be construed as limiting.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

Certain terminology is used herein for convenience only. For example, words such as “top”, “bottom”, “upper,” “lower,” “left,” “right,” “horizontal,” “vertical,” “upward,” and “downward” merely describe the configuration shown in the figures or the orientation of a part in the installed position. Indeed, the referenced components may be oriented in any direction. Similarly, throughout this disclosure, where a process or method is shown or described, the method may be performed in any order or simultaneously, unless it is clear from the context that the method depends on certain actions being performed first.

A coordinate system is presented in the Figures and referenced in the description in which the “Y” axis corresponds to a vertical direction, the “X” axis corresponds to a horizontal or lateral direction, and the “Z” axis corresponds to the interior/exterior direction.

### DESCRIPTION OF VARIOUS EMBODIMENTS

The present disclosure relates to non-mammalian biointerfaces used as a substrate or as a part of a substrate for retention, culture, and/or growth of non-mammalian cells and viruses (e.g., for retaining and maintaining algal spores and growing mature seaweed therefrom), and related systems, methods, and apparatuses. In various examples, the non-mammalian biointerface is operable as a substrate for growth of multi-cellular non-mammalian organisms (e.g., seaweed, mushrooms).

In the instant disclosure, the examples are primarily described in association with retention of algal spores and growth of algae therefrom, although it should be readily appreciated feature of such examples are equally applicable to other non-mammalian cells including, for example, plant cells, insect cells, bacterial cells, yeast cells, as well as viruses. Non-mammalian biointerfaces according to the instant disclosure can be used in a variety of applications, including non-mammalian cell capture, non-mammalian cell culture and growth, non-mammalian cell and/or tissue transport and deposition, and 3-dimensional (3D) non-mammalian cell and/or tissue culture, for example. In some embodiments, non-mammalian biointerfaces according to the disclosure can be used in bioreactors or synthetic biology applications.

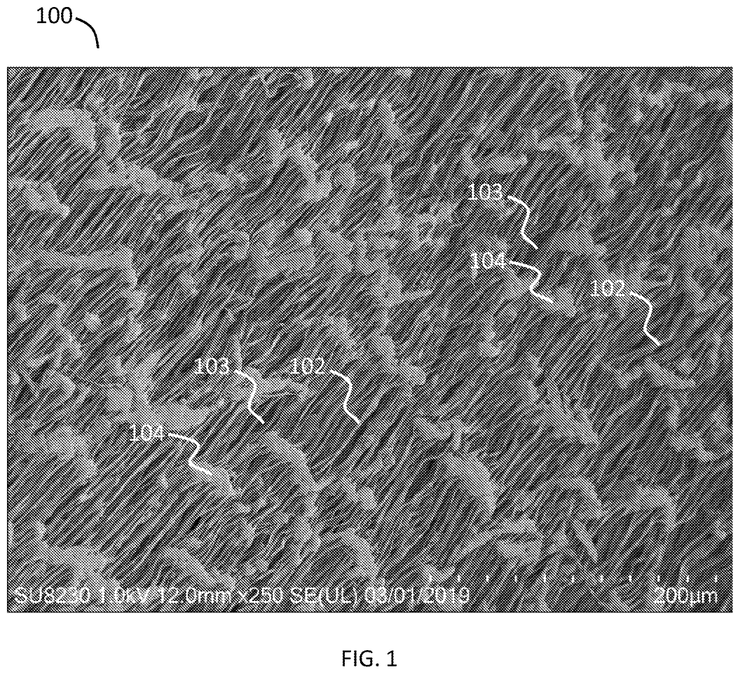

In some embodiments, the non-mammalian biointerface includes a fibrillated material having a microstructure including a plurality of fibrils defining an average inter-fibril distance. FIG. 1 is an SEM micrograph depicting a microstructure 100 of a non-mammalian biointerface including a fibrillated material according to some embodiments. The fibrillated material depicted in FIG. 1 having the microstructure 100 is expanded polytetrafluoroethylene (ePTFE). As depicted, the microstructure 100 is defined by a plurality of fibrils 102 that interconnect nodes 104. The fibrils 102 define inter-fibril spaces 103.

The fibrils 102 have a defined average inter-fibril distance, which in some embodiments may be from about 1 μm to about 200 μm, from about 1 μm to about 50 μm, from about 1 μm to about 20 μm, from about 1 μm to about 10 μm, from about 1 μm to about 5 μm, from about 5 μm to about 50 μm, from about 5 μm to about 20 μm, from about 5 μm to about 10 μm, from about 10 μm to about 100 μm, from about 10 μm to about 75 μm, from about 10 μm to about 50 μm, from about 10 μm to about 25 μm, from about 25 μm to about 200 μm, from about 25 μm to about 150 μm, from about 25 μm to about 100 μm, from about 25 μm to about 50, from about 50 μm to about 200 μm, from about 50 μm to about 150 μm, from about 50 μm to about 100 μm, from about 100 μm to about 200 μm, from about 100 μm to about 150 μm, or from about 150 μm to about 200 μm. In some embodiments, the fibrils 102 may have an average inter-fibril distance of about 1 μm, about 2 μm, about 3 μm, about 4 μm, about 5 μm, about 10 μm, about 20 μm, about 30 μm, about 40 μm, about 50 μm, about 60 μm, about 70 μm, about 80 μm, about 90 μm, about 100 μm, about 110, about 120 μm, about 130 μm, about 140 μm, about 150 μm, about 160 μm, about 170 μm, about 180 μm, about 190 μm, or about 200 μm.

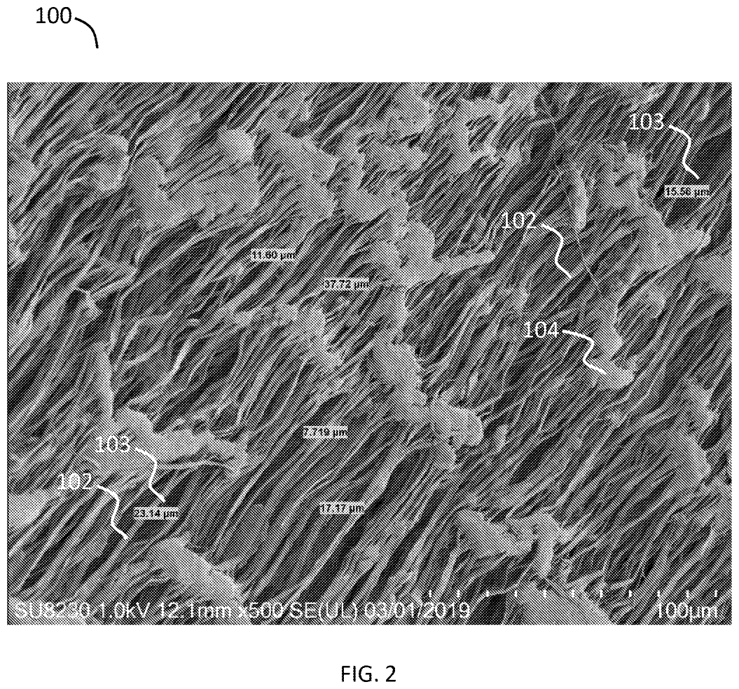

FIG. 2 is a higher magnification SEM micrograph of the microstructure depicted in FIG. 1. FIG. 2 identifies the dimension of select inter-fibril spaces 103 in μm.

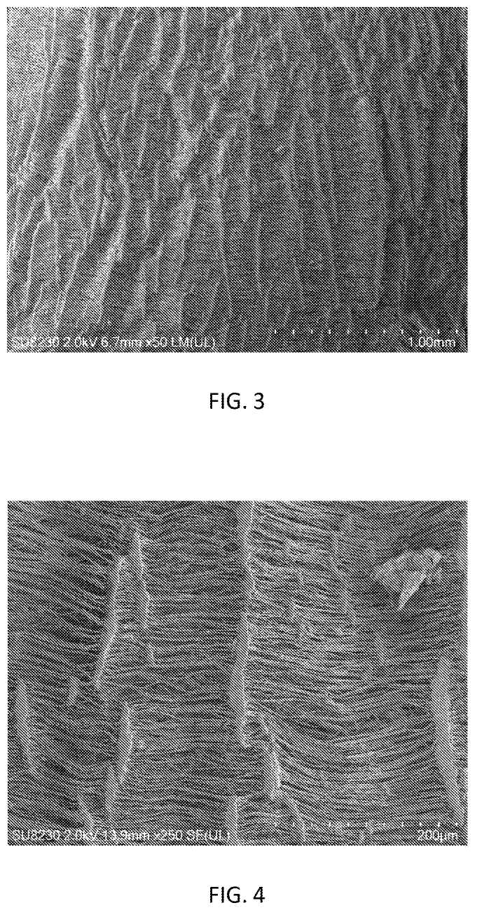

FIG. 3 is an SEM micrograph depicting another microstructure of a non-mammalian biointerface that includes a fibrillated ePTFE material according to some embodiments.

FIG. 4 is a higher magnification SEM micrograph of the microstructure depicted in FIG. 3.

At least some of the fibrils 102 are sufficiently spaced from each other to retain a non-mammalian cell or virus in an inter-fibril space 103.

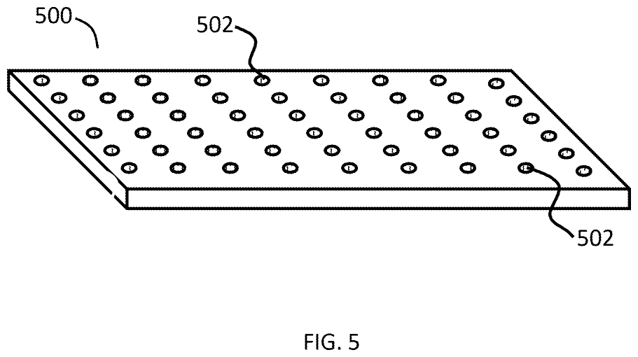

FIG. 5 is a perspective view of a schematic representation of the microstructure of a non-mammalian biointerface according to some embodiments. As depicted, the microstructure 500 is defined by a plurality of pores 502.

The pores 502 may be round, approximately round, or oblong. The pores 502 may have a diameter or approximate diameter from about 1 μm to about 200 μm, from about 1 μm to about 50 μm, from about 1 μm to about 20 μm, from about 1 μm to about 10 μm, from about 1 μm to about 5 μm, from about 5 μm to about 50 μm, from about 5 μm to about 20 μm, from about 5 μm to about 10 μm, from about 10 μm to about 100 μm, from about 10 μm to about 75 μm, from about 10 μm to about 50 μm, from about 10 μm to about 25 μm, from about 25 μm to about 200 μm, from about 25 μm to about 150 μm, from about 25 μm to about 100 μm, from about 25 μm to about 50, from about 50 μm to about 200 μm, from about 50 μm to about 150 μm, from about 50 μm to about 100 μm, from about 100 μm to about 200 μm, from about 100 μm to about 150 μm, or from about 150 μm to about 200 μm. In some embodiments, the pores 502 may have a diameter or approximate diameter of about 1 μm, about 2 μm, about 3 μm, about 4 μm, about 5 μm, about 10 μm, about 20 μm, about 30 μm, about 40 μm, about 50 μm, about 60 μm, about 70 μm, about 80 μm, about 90 μm, about 100 μm, about 110, about 120 μm, about 130 μm, about 140 μm, about 150 μm, about 160 μm, about 170 μm, about 180 μm, about 190 μm, or about 200 μm.

In some embodiments, the inter-fibril spaces 103 of FIG. 1 form the pores 502 of FIG. 5. That is, a microstructure 100 having a plurality of fibrils 102 may form the porous microstructure 500. However, not all microstructures 500 having pores 502 are fibrillated.

The microstructure of the non-mammalian biointerface is configured to retain viruses or non-mammalian cells. In some embodiments, the microstructure is configured to retain algal cells, algal spores, algal gametophytes and/or sporophytes, plant cells, plant spores, seedlings, insect cells, bacterial cells, bacterial endospores, yeast cells, fungal cells, fungal spores, viruses, or a combination thereof. In some embodiments, the non-mammalian biointerface retains a plurality of non-mammalian cells. The plurality of non-mammalian cells may all be of the same cell type, or of two or more different cell types. In some embodiments, the non-mammalian biointerface retains two different cell types that display a symbiotic relationship when cultured or grown together. For example, growth of a terrestrial plant and symbiotic mycorrhizae may be supported on the non-mammalian biointerface. For sake of simplicity, throughout this disclosure reference will be made to “non-mammalian cells,” although viruses, spores, endospores, gametophytes, sporophytes, and seedlings are also contemplated by this term and are considered to be within the purview of the disclosure.

In some embodiments, in addition to retaining non-mammalian cells, non-mammalian biointerfaces of the instant disclosure promote growth and/or proliferation of the retained non-mammalian cells. That is, the non-mammalian biointerface viably maintains the retained non-mammalian cells. The non-mammalian biointerface creates a microenvironment conducive to the growth and/or proliferation of the retained non-mammalian cells.

In certain embodiments, the non-mammalian biointerface creates a selective microenvironment conducive to the growth and/or proliferation of a target non-mammalian cell while inhibiting or preventing growth and/or proliferation of non-target non-mammalian cells. A selective microenvironment can be achieved by, for example, providing a combination of inter-fibril distance and/or pore size, material density, ratio of inter-fibril distance to average density of material, depth or thickness, hydrophobicity, and presence or absence of nutrient sources, moisture, bioactive agents, and adhesives that supports growth and/or proliferation of the target non-mammalian cells while inhibiting or preventing growth and/or proliferation of non-target non-mammalian cells.

Several factors may affect retention and/or viable maintenance of the non-mammalian cells. Such factors include, for example, the inter-fibril distance and/or pore size, material density, a ratio of inter-fibril distance to average density of material, depth or thickness, hydrophobicity, and presence or absence of nutrient sources, moisture, bioactive agents, and adhesives. These factors will each be described in more detail.

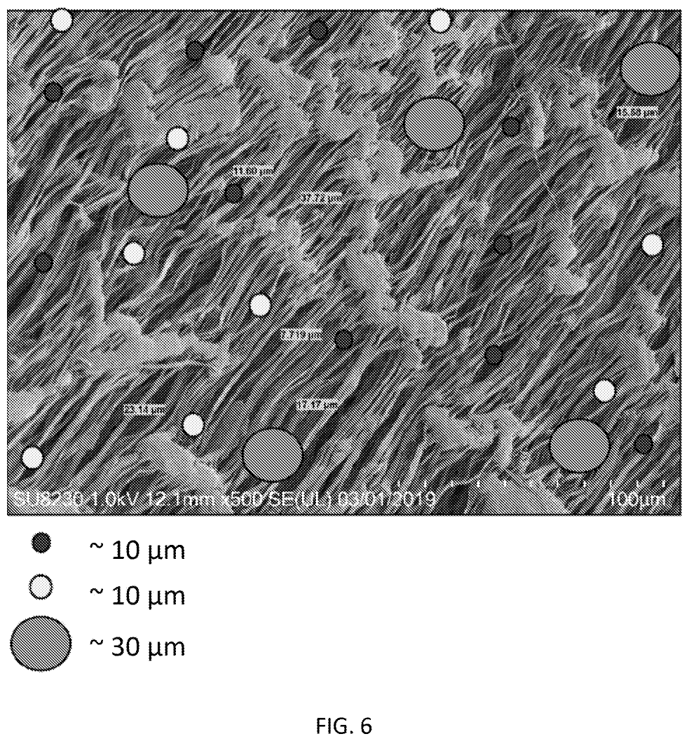

The distance between two fibrils (i.e., inter-fibril distance) defines an inter-fibril space 103. In some embodiments, an inter-fibril space 103—and thus the inter-fibril distance—is sufficient to retain a non-mammalian cell therein; the cell is retained between the two fibrils defining the inter-fibril space. The inter-fibril distance is sufficient to allow at least a portion of the non-mammalian cell to enter between the two fibrils defining the inter-fibril space 103. In some embodiments, the non-mammalian cell is thereby retained within the microstructure of the non-mammalian biointerface. FIG. 6 is a modified version of the photograph of FIG. 2, depicting a microstructure of a non-mammalian biointerface including a fibrillated material and overlaid with representative non-mammalian cells having a diameter of either about 10 μm or about 30 μm. FIG. 6 illustrates how and where target non-mammalian cells may enter between the two fibrils defining an inter-fibril space.

In some embodiments, the average inter-fibril distance is controlled in order to encourage ingress of at least portions of target non-mammalian cells into the microstructure. For example, where it is desirous for the microstructure to retain spores of dulse (Palmaria palmata), which have a diameter of about 30 μm, the average inter-fibril distance of the microstructure is about 30 μm, or slightly larger (e.g., about 32 μm to about 35 μm). In some embodiments, target non-mammalian cells have a diameter of about 0.5 μm to about 200 μm.

In some embodiments, about half of the target non-mammalian cell may enter the inter-fibril space 103. In such embodiments, the inter-fibril distance is at least equal to a dimension (e.g., diameter or width) of the target non-mammalian cell. In some embodiments, the inter-fibril distance is slightly larger than the dimension of the target cell. This allows for the entire spore to enter the inter-fibril space 103 and be retained therein.

In some embodiments, more than half of the target non-mammalian cell may enter the inter-fibril space 103, up to the entire cell. In such embodiments, the portion of the cell entering the inter-fibril space 103 may be governed by the depth of a pore, the opening of which is defined by the inter-fibril space. The depth of the pore may be controlled by, for example, material density.

In some embodiments, only a portion of the non-mammalian cell enters the inter-fibril space 103. Therefore, where the inter-fibril distance is less than the diameter of the target non-mammalian cell, the target non-mammalian cell may only partially enter the inter-fibril space 103. Where the target non-mammalian cell only partially enters the inter-fibril space 103, the target non-mammalian cell may none-the-less be retained therein if a sufficient portion of the target non-mammalian cell enters the inter-fibril space 103. In some embodiments, a substance such as an adhesive applied to the microstructure may reduce the portion of the cell required to enter the inter-fibril space 103 to aid in retention.

In some embodiments, the microstructure is formed by a non-fibrillated material. In certain embodiments, the pore openings 502 are inherent to the material of the cultivation substrate. It will be recognized that different materials may have different pore opening properties, and that a material may be manufactured or otherwise manipulated to provide the desired pore opening properties. In other embodiments, the pore openings 502 are formed by micro drilling techniques such as, for example: mechanical micro drilling, such as ultrasonic drilling, powder blasting or abrasive water jet machining (AWJM); thermal micro drilling, such as laser machining; chemical micro drilling, including wet etching, deep reactive ion etching (DRIE) or plasma etching; and hybrid micro drilling techniques, such as spark-assisted chemical engraving (SACE), vibration-assisted micromachining, laser-induced plasma micromachining (LIPMM), and water-assisted micromachining.

In those embodiments where the microstructure is formed by a non-fibrillated material, the pore openings 502 act much like the inter-fibril spaces 103 described and are of a sufficient size to allow at least a portion of a target non-mammalian cell to enter the pore opening 502. In some embodiments, the non-mammalian cell is thereby retained within the microstructure of the non-mammalian biointerface. In some embodiments, the size of pore openings 502 is controlled to encourage ingress of a least portions of target non-mammalian cells into the microstructure. For example, where it is desirous for the microstructure to retain spores of dulse (Palmaria palmata), which have a diameter of about 30 μm, the pore openings 502 of the microstructure have a diameter of about 30 μm, or slightly larger (e.g., about 32 μm to about 35 μm). In some embodiments, target non-mammalian cells have a diameter of about 0.5 μm to about 200 μm.

In some embodiments, about half of the target non-mammalian cell may enter the pore opening 502. In such embodiments, the pore opening is at least equal to a dimension (e.g., diameter or width) of the target non-mammalian cell. In some embodiments, the pore opening is slightly larger than the dimension of the target cell. This allows for the entire spore to enter the pore opening 502 and be retained therein.

In some embodiments, more than half of the target non-mammalian cell may enter the pore opening 502, up to the entire cell. In such embodiments, the portion of the cell entering the pore opening 502 may be governed by the pore depth of a pore. The depth of the pore may be controlled by, for example, material density.

In some embodiments, only a portion of the non-mammalian cell enters the pore opening 502. Therefore, where the pore opening is smaller than the diameter of the target non-mammalian cell, the target non-mammalian cell may only partially enter the pore opening 502. Where the target non-mammalian cell only partially enters the pore opening 502, the target non-mammalian cell may none-the-less be retained therein when a sufficient portion of the target non-mammalian cell enters the pore opening. In some embodiments, a substance such as an adhesive applied to the microstructure may reduce the portion of the cell required to enter the pore opening 502 to aid in retention.

In some embodiments, the non-mammalian biointerface includes a low-density material. The low-density material may be fibrillated or non-fibrillated, and in some embodiments, defines the microstructure of the non-mammalian biointerface. The density of the low-density material may be about 0.1 g/cm³, about 0.2 g/cm³, about 0.3 g/cm³, about 0.4 g/cm³, about 0.5 g/cm³, about 0.6 g/cm³, about 0.7 g/cm³, about 0.8 g/cm³, about 0.9 g/cm³, or about 1.0 g/cm³. In some embodiments, the density of the low-density material is from about 0.1 0.1 g/cm³ to about 1 g/cm³.

In some embodiments, the low-density material provides a sufficient pore depth to retain non-mammalian cells in either inter-fibril spaces 103 or pore openings 502.

In some embodiments, the dimensions of the pore openings (length (μm) and width (μm)), whether formed by a fibrillated or non-fibrillated material, together with the depth at which target non-mammalian cells enter the pores (μm) define a capture ratio. Each cell type may have a different capture ratio required for adequate retention of cells by the microstructure. The required capture ratio may be influenced by the properties of the material making up the microstructure and the presence or absence of nutrients, adhesives, and/or bioactive agents.

In some embodiments, the low-density material allows the non-mammalian cells to proliferate or otherwise grow into the low-density material. For example, as dulse spores retained in a low-density material having a microstructure described herein develop into gametophytes and sporophytes, the dulse grows into the low-density material in all three dimensions (i.e., horizontally in x- and y-dimensions and depth-wise in the z-dimension). This three-dimensional growth allows for improved retention of the dulse gametophytes and sporophytes.

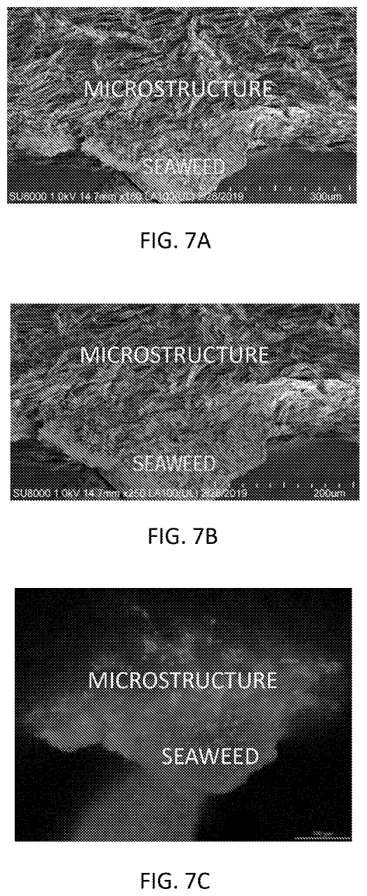

FIGS. 7A and 7B are cross-sectional SEM micrographs taken at two different magnifications of a low-density microstructured material according to some embodiments, depicting dulse seaweed ingrowth into the low-density material. FIG. 7C is a cross-sectional micrograph generated using optical fluorescence microscopy depicting dulse seaweed ingrowth into the low-density material.

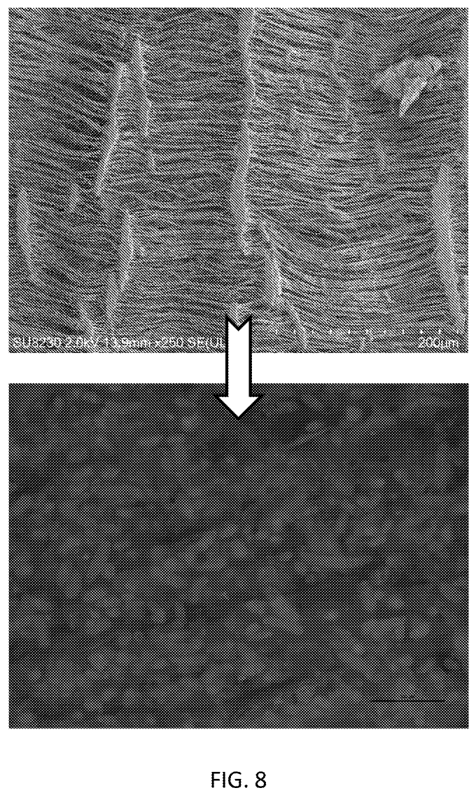

FIG. 8 (top panel) is an SEM micrograph of the surface of a low density microstructured material according to some embodiments. FIG. 8 (bottom panel) depicts the same culture substrate material as the top panel following seeding with sugar kelp spores and germination thereof.

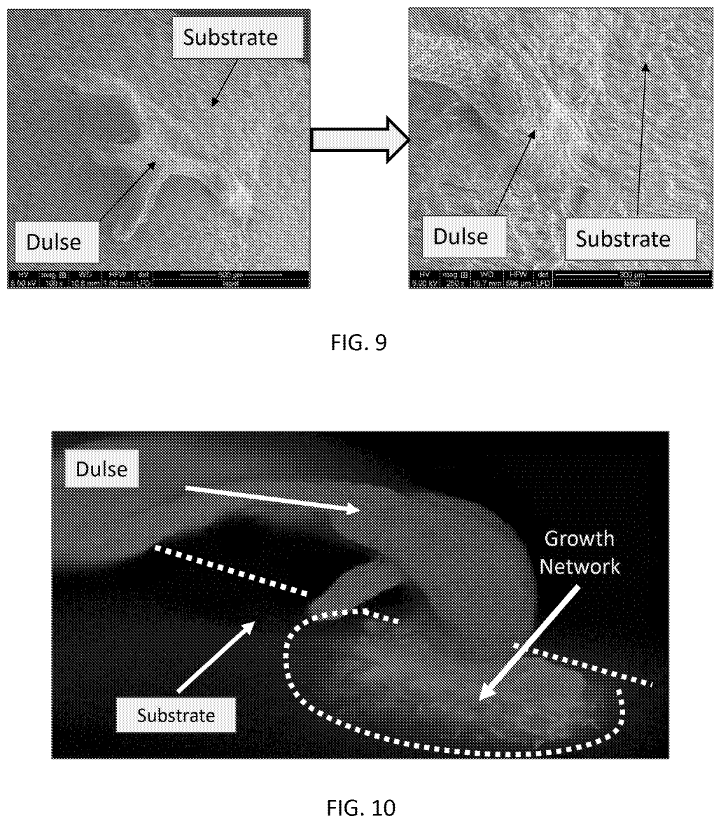

FIG. 9 depicts SEM micrographs of the surface of a microstructure taken at two different magnifications, where dulse seaweed can clearly be seen to be attached and to and growing into the microstructure. FIG. 10 depicts a fluorescence microscopy micrograph of the surface of a microstructure to which the dulse seaweed is attached and growing into the microstructure. The seaweed growth is observed to be growing into the microstructure in a ‘growth network’, securely anchoring the seaweed to the microstructure

It is evident from the micrographs of FIGS. 7A-FIG. 10 that the dulse seaweed is able to grow into the microstructure of the fibrillated ePTFE in all three dimensions.

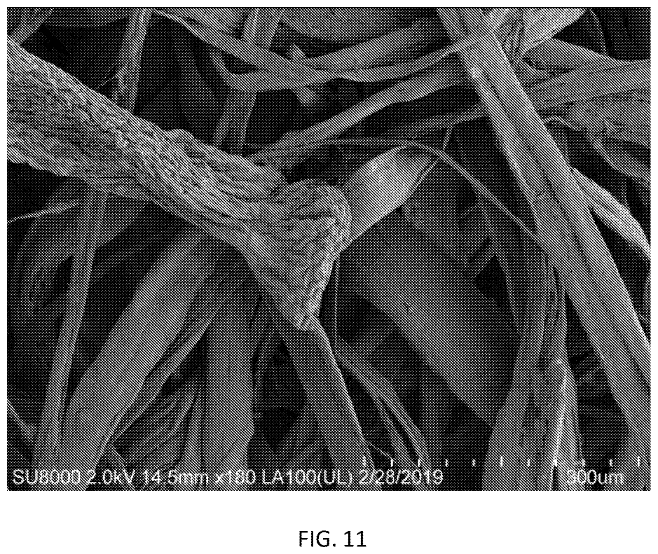

Conversely, FIG. 11 is a photograph depicting dulse seaweed growing on the surface of a higher-density fibrillated material. The dulse is unable to grow into the higher-density material, and rather attaches solely to the fibrils at the material's surface. This results in weaker retention of the dulse gametophyte relative to the low-density material, in which the developing dulse gametophyte becomes anchored.

In some embodiments, the non-mammalian cells grow and or proliferate deep into the microstructure. This deep ingrowth and incorporation into the microstructure gives additional benefits in protecting the non-mammalian cells from external environments (e.g., in the case of seaweed gametophytes, the sea). In some embodiments, the depth of penetration of the non-mammalian cells relative to the initial size of the non-mammalian cells is from about 1:1 to about 200:1. For example, for a dulse spore having an initial diameter of about 30 μm, the dulse gametophyte may grow into the microstructure to a depth of about 30 μm to about 6 mm.

In some embodiments, the low-density material has a thickness sufficient to allow for a desired level of ingrowth. In some embodiments, the non-mammalian biointerface includes a single layer of the low-density material. In some embodiments, the non-mammalian biointerface includes two or more layers of the low-density material. In certain embodiments, the two or more layers are present in a laminate, i.e., a laminate of a plurality of layers of the low-density material.

In some embodiments, the inter-fibril distance and the density of the material having a microstructure defines a ratio of the average inter-fibril distance (μm) to the average density (g/cm³) of the fibrillated material. In some embodiments, the ratio of the average inter-fibril distance (μm) to the average density (g/cm³) of the fibrillated material may be about 1:1, about 10:1, about 20:1, about 30:1, about 40:1, about 50:1, about 60:1, about 70:1, about 80:1, about 90:1, about 100:1, about 125:1, about 150:1, about 175:1, about 200:1, about 225:1, about 250:1, about 275:1, about 300:1, about 325:1, about 350:1, about 375:1, about 400:1, about 425:1, about 450:1, about 475:1, about 500:1, about 550:1, about 600:1, about 650:1, about 700:1, about 750:1, about 800:1, about 900:1, about 1000:1, about 1250:1, about 1500:1, about 1750:1, or about 2000:1. In some embodiments, the ratio of the average inter-fibril distance (μm) to the average density (g/cm³) of the fibrillated material is from about 1:1 to about 2000:1.

In some embodiments, the non-mammalian biointerface includes one or more adhesives. An adhesive may be applied to the surface of the microstructure, imbibed within the microstructure, or both applied to the surface and imbibed within the microstructure. In some embodiments, the adhesive includes one or more cell-adhesive ligands specific to the target non-mammalian cell(s) to be retained by the non-mammalian biointerface.

In some embodiments, a non-mammalian biointerface described herein includes a nutrient phase associated with at least a portion of the non-mammalian biointerface. The nutrient phase serves to viably maintain the non-mammalian cells retained by the non-mammalian biointerface. In some embodiments, the nutrient phase promotes growth and/or proliferation of the retained non-mammalian cells within the microstructure. In some embodiments, the nutrient phase acts to maintain and/or encourage attachment to and ingrowth into or integration within the microstructure.

In some embodiments, the nutrient phase acts as a chemoattractant capable of attracting the non-mammalian cells to predetermined locations of the non-mammalian biointerface to which the nutrient phase is applied or included.

The nutrient phase can be located within the microstructure of the non-mammalian biointerface, on the microstructure (e.g., on its surface), or located both within and on the microstructure. In some embodiments, the nutrient phase is applied to a surface of the non-mammalian biointerface as a coating. In some embodiments, the nutrient phase is included within the material forming the microstructure. Where the nutrient phase is included within the material forming the microstructure, the nutrient phase may encourage ingrowth into or integration within the microstructure.

In some embodiments, the nutrient phase includes at least one nutrient beneficial to the target non-mammalian cells to be retained by the biointerface. For example, where seaweed spores are to be retained by the microstructure, the nutrient phase can include macronutrients (e.g., nitrogen, phosphorous, carbon, etc.), micronutrient (e.g., iron, zinc, copper, manganese, molybdenum, etc.), and vitamins (e.g., vitamin B₁₂, thiamine, biotin). The nutrients of the nutrient phase can be provided in various forms. For example, nitrogen can be provided as ammonium nitrate (NH₄NO₃), ammonium sulfate ((NH₄)₂SO₄), calcium nitrate (Ca(NO₃)₂), potassium nitrate (KNO₃), urea (CO(NH₂)₂), etc. It will be recognized by those of skill in the art which nutrients would be beneficial to include in the nutrient phase so as to viably maintain the non-mammalian cells to be retained by the biointerface.

Which nutrients to include in the nutrient phase will depend on which cells are to be retained by the biointerface, as various cell types will have different nutrient needs, as well as the intended use of the biointerface. For example, where a non-mammalian biointerface retaining non-mammalian cells is to be introduced into an environment that is deficient in essential nutrients, all nutrients required by the cells can be included in the nutrient phase. Where a non-mammalian biointerface retaining non-mammalian cells is to be introduced into an environment having at least one essential nutrient, those environmentally-available essential nutrients may be excluded from the nutrient phase or included at a lower concentration. The biointerface may also act to concentrate nutrients from the environment by capturing the environmental nutrients in the microstructure. This may be advantageous in environments where environmental nutrients are present only in low concentrations.

In some embodiments, and as further described elsewhere herein, the biointerface can be used to transport retained cells from location to another. Where the biointerface functions as a transportation medium, the nutrient phase may include sufficient nutrient levels to viably support the retained cells during transport. In some embodiments the nutrient phase may include sufficient nutrient levels to viably maintain the retained cells post-transport, following introduction of the retained cells into a new environment.

In some embodiments the nutrient phase includes one or more carriers. Carriers can include, for example, liquid carriers, gel carriers, and hydrogel carriers. In some embodiments, a carrier of the nutrient phase is an adhesive. Including an adhesive as a carrier of the nutrient phase can function to ensure that the nutrient phase remains on and/or within the biointerface. Where the nutrient phase is applied to a surface of the biointerface and includes an adhesive as a carrier, the nutrient face may also function to promote retention of non-mammalian cells within the microstructure.

In some embodiments, the nutrient phase is formulated to control release rates of the nutrients.

In some embodiments, the biointerface further comprises a salt associated with the microstructure. In some embodiments, the salt is sodium chloride (NaCl). Salt associated with the microstructure can produce and maintain a saline microenvironment for the retained non-mammalian cells. This can be particularly advantageous where non-mammalian marine cells (e.g., seaweed, marine plants) are retained by the biointerface. In some embodiments, a saline microenvironment within the biointerface can be maintained when the biointerface is submerged in fresh water, thereby viably maintaining non-mammalian marine cells and avoiding the need to maintain a saline culture environment, which can be difficult and costly.

In some embodiments, the biointerface includes a liquid-containing phase associated with at least a portion of the non-mammalian biointerface. The liquid-containing phase serves to provide and maintain moisture within the microstructure's microenvironment, which may be beneficial to the viable maintenance of the non-mammalian cells retained therein.

In some embodiments, the biointerface includes a liquid wicking material. The liquid wicking material can be the same material that forms the microstructure. The liquid wicking material functions to maintain moisture within the microstructure's microenvironment.

While spores and endospores may be viably maintained in an arid environment, the non-mammalian cells will generally require moisture to grow and/or proliferate. By maintaining a moist microenvironment (e.g., by including a liquid-containing substrate and/or a liquid wicking material), it may be possible to transport the biointerface having non-mammalian cells retained therein without having to maintain the biointerface in an aqueous environment.

In some embodiments, the liquid containing phase is entrained within the microstructure, entrained on the microstructure, or entrained both within and on the microstructure. In some embodiments, the liquid containing phase is present as a coating on a surface of the non-mammalian biointerface.

In some embodiments, the liquid containing phase includes, for example, a hydrogel, a slurry, a paste, or a combination of a hydrogel, a slurry, and/or a paste. In some embodiments, the liquid containing phase is a carrier for the nutrient phase.

In some embodiments, at least a portion of the non-mammalian biointerface is hydrophilic. Such hydrophilic portions of the non-mammalian biointerface may contribute to the microstructure's ability to retain the non-mammalian cells.

In some embodiments, at least a portion of the non-mammalian biointerface is hydrophobic. Such hydrophobic portions of the non-mammalian biointerface may reduce or prevent retention of non-mammalian cells, and may help reduce or prevent biofouling and attachment of unwanted cells.

In some embodiments, one or more portions of the non-mammalian biointerface is hydrophobic and one or more portions of the non-mammalian biointerface is hydrophilic, such that the non-mammalian cells are selectively encouraged to be retained in the one or more hydrophilic portions of the non-mammalian biointerface.

In some embodiments, the non-mammalian biointerface may include one or more bioactive agents associated with the non-mammalian biointerface. Bioactive agents include any agent having an effect, whether positive or negative, on the cell or organism coming into contact with the agent. Suitable bioactive agents may include, for example, biocides and serums. Biocides may be associated with portions of the microstructure to prevent attachment and growth of unwanted cells or organisms to those portions of the microstructure. Unwanted cells may include non-target non-mammalian cells such as bacteria, yeast, and algae, for example. Biocides may also deter pests, such as insects. In some embodiments, the biocide prevents attachment and growth of the target non-mammalian cell to portions of the biointerface where attachment and growth is not desired. In some embodiments, serums may be applied to portions of the biointerface. Serums may aid in cell attachment and retention and/or encourage cell growth and proliferation. Serums may include cell-adhesive ligands, for example, as well as provide a source of growth factors, hormones, and attachment factors.

In some embodiments, the microstructure of the non-mammalian biointerface is patterned. By specifically patterning the microstructure, it is possible to specifically retain target non-mammalian cells at described portions of the microstructure while excluding cells from other portions.

In some embodiments, the microstructure includes a pattern of higher density portions and lower density portions. In such a configuration, the lower density portions correspond to a portion of the microstructure configured to retain and viably maintain the target non-mammalian cells, while the higher density portions inhibit or prevent retention of non-mammalian cells. The density pattern may extend in any dimension. For example, a high-density/low-density pattern may extend in the x- or y-dimension of the non-mammalian biointerface, or in the z-dimension. When extending in the z-dimension, the outermost portion will generally be a lower density portion configured to retain and viably maintain the non-mammalian target cells. Underlying portions may be of a higher density, or may be of an even lower density than the outermost portion. Where the underlying portion is of a higher density, ingrowth of the non-mammalian cells will be inhibited or prevented. Where the underlying portion is of a lower density than the outermost portion, ingrowth of the non-mammalian cells will be encouraged and/or facilitated. In some embodiments, the density pattern or gradient in the z-dimension results from concentric wraps of microstructure material having differing densities, or from a laminate configuration in which each lamina has a different density. In some embodiments, the density pattern can extend in two or all three dimensions. In some embodiments, portions of the microstructure have a density gradient.

Density can be measured in various ways, including, for example, measuring dimensions and weight of the material. In addition, wetting experiments can be conducted to derive density values. Density can be modified by, for example, altering inter-fibril distance, number of fibrils per unit volume, number of pores per unit volume, and pore size.

In some embodiments, the lower density portions are characterized by a material density of about 1.0 g/cm³ or less, whereas the higher density portions are characterized by a density of about 1.7 g/cm³ or greater. As depicted by FIGS. 5A-5C and 6, attachment and retention of non-mammalian cells (dulse seaweed depicted) can be significantly affected by microstructure material density, with the lower density material (i.e., about 1.0 g/cm³ or less) demonstrating improved ingrowth and retention.

In some embodiments, the density is that of the material itself that forms the microstructure; i.e., does not have any inclusions such as a nutrient phase, liquid containing phase, etc.

In some embodiments, the density is that of the material and an inclusion such as a nutrient phase, a liquid containing phase, or a density-altering filler. In some embodiments, portions of the microstructure are filled with a filler to alter the density, thereby altering the ability of that portion of the microstructure to retain non-mammalian cells and/or prevent ingrowth into the microstructure.

In some embodiments, the non-mammalian biointerface includes a material having a pattern of higher porosity portions and lower porosity portions. In some embodiments, the lower porosity portions correspond to portions of the microstructure configured to retain and viably maintain the target non-mammalian cells. In some embodiments, the higher porosity portions correspond to portions of the microstructure configured to retain and viably maintain the target non-mammalian cells.

In some embodiments, the non-mammalian biointerface includes a pattern of greater inter-fibril distance portions and lower inter-fibril distance portions. In some embodiments, the lower inter-fibril distance portions correspond to the portions of the microstructure configured to retain and viably maintain the non-mammalian cells. In such embodiments, the higher inter-fibril distance portions have inter-fibril distances too great to retain the target non-human cells. In some embodiments, the higher inter-fibril distance portions correspond to the portions of the microstructure configured to retain and viably maintain the non-mammalian cells. In such embodiments, the lower inter-fibril distance portions have inter-fibril distances too small to retain the target non-mammalian cells.

In some embodiments, the pattern of the patterned cultivation substrate is generated by controlling at least two of density, porosity, and average inter-fibril distance. In some embodiments, the pattern of the patterned non-mammalian biointerface, whether involving density, porosity, average inter-fibril distance, or a combination thereof, may be an organized or selective pattern, or may be a random pattern.

In some embodiments, the pattern can be set or adjusted by selective application of longitudinal tension. Setting or adjusting the pattern by application of longitudinal tension allow for one to alter the pattern mechanically. In some embodiments, a pattern is set or adjusted in fibrillated material by selective application of longitudinal tension.

In some embodiments, a patterned non-mammalian biointerface includes portions that have two or more characteristics favorable to non-human cell retention. For example, a patterned non-human mammalian biointerface can have portions of low-density (i.e., about 1.0 g/cm³ or less) and an average inter-fibril distance selected to retain the target non-mammalian cells (e.g., about 30 μm for dulse spores). These same portions may further be hydrophilic and/or include one or more of a nutrient phase, an adhesive, and a bioactive agent. The density, inter-fibril distance, hydrophobicity, nutrient phase, adhesive, and bioactive agent, for example, may each be selected to preferentially retain a target non-mammalian cell or cells.

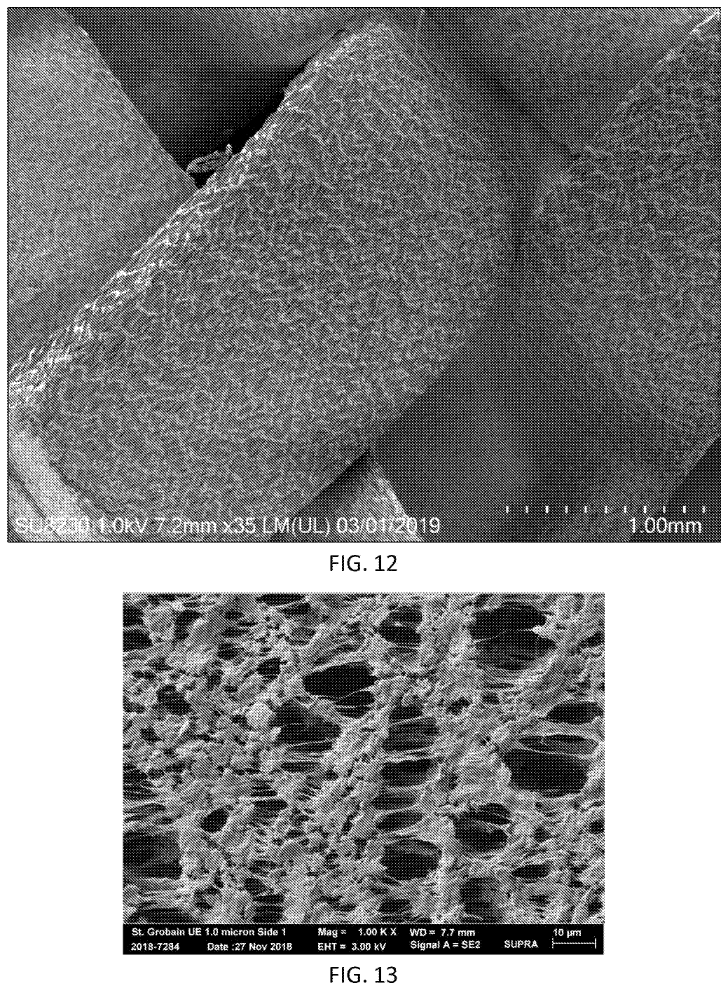

In some embodiments, the non-mammalian biointerface is configured as a fiber, a membrane, a woven article, a non-woven article, a braided article, a fabric, a knit article, a particulate dispersion, or combinations of these. FIG. 12 is a photograph of a non-mammalian biointerface according to certain embodiments, where the biointerface is configured as a woven article. As demonstrated by FIG. 12, each strand of the woven article comprises a microstructure. In such a configuration, not only can target non-mammalian cells grow and proliferate through the depth of the strand, but can also grow and proliferate in the spaces between the woven strands. In the example of the dulse seaweed, this can provide for additional mechanical retention capacity as the seaweed grows around the woven strands.

In some embodiments, the non-mammalian biointerface includes at least one of a backer layer, a carrier layer, a laminate of a plurality of layers, a composite material, or combinations of these. The microstructure of the biointerface can be deposited on the backer layer or carrier layer, or included in a laminate. The backer layer can be, for example, a rope or metal cable. For example, where the non-mammalian biointerface is to retain and viably maintain seaweed spores, the biointerface can be deposited on a rope or metal cable to produce a seed rope, eliminating the need to wrap a seed string around a rope in the field for open water rope cultivation of seaweed.

In some embodiments, the material having the microstructure itself has sufficient strength to be moved as a conveyor belt through various growth stages of the retained non-mammalian cells, including harvest of the non-mammalian cells. In some embodiments, the material having the microstructure is deposited on a backer layer, carrier layer, or formed into a laminate to produce a biointerface having sufficient strength to be moved as a conveyor belt through various growth stages of the retained non-mammalian cells, including harvest of the non-mammalian cells.

In some embodiments, the non-mammalian biointerface is configured as a particulate dispersion. The microstructure is provided by a plurality of particles in a dispersion formulated for deposition onto a backer layer or a carrier substrate to form the non-mammalian biointerface. The particles can be, for example, shredded or otherwise fragmented pieces of a fiber, a membrane, a woven article, a non-woven article, a braided article, a fabric, or a knit article having a microstructure as described herein. In some embodiments, non-mammalian cells are contacted with the particles prior to deposition onto a backer layer or carrier substrate. In other embodiments, non-mammalian cells are contacted with the particles following deposition onto the backer layer or carrier substrate. The particulate dispersion may be deposited onto the backer layer or carrier substrate by, for example, spraying, dip-coating, brushing, or other coating means. In embodiments in which cells are retained in the microstructure of the particles prior to deposition, care must be taken to ensure that the deposition method does not negatively affect the retained cells. Certain non-mammalian cells, such as spores and endospores, may be more resilient and capable of withstanding deposition.

In some embodiments, the non-mammalian biointerface comprises an expanded fluoropolymer. In some embodiments, the expanded fluoropolymer forms the microstructure of the non-mammalian biointerface. In some embodiments, the expanded fluoropolymer is selected from the group of expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), expanded polytetrafluoroethylene (ePTFE), and modified ePTFE. Examples of suitable expanded fluoropolymers include fluorinated ethylene propylene (FEP), porous perfluoroalkoxy alkane (PFA), polyester sulfone (PES), poly (p-xylylene) (ePPX) as taught in U.S. Patent Publication No. 2016/0032069, ultra-high molecular weight polyethylene (eUHMWPE) as taught in U.S. Pat. No. 9,926,416 to Sbriglia, ethylene tetrafluoroethylene (eETFE) as taught in U.S. Pat. No. 9,932,429 to Sbriglia, polylactic acid (ePLLA) as taught in U.S. Pat. No. 7,932,184 to Sbriglia, et al., vinylidene fluoride-co-tetrafluoroethylene or trifluoroethylene [VDF-co-(TFE or TrFE)] polymers as taught in U.S. Pat. No. 9,441,088 to Sbriglia

In some embodiments, the expanded fluoropolymer includes the nutrient phase. This may be achieved by co-blending the nutrient phase with the fluoropolymer resin prior to extrusion and expansion of the fluoropolymer.

In some embodiments, the non-mammalian biointerface comprises an expanded thermoplastic polymer. In some embodiments, the expanded thermoplastic polymer forms the microstructure of the non-mammalian biointerface. In some embodiments, the expanded thermoplastic polymer is selected from the group of expanded polyester sulfone (ePES), expanded ultra-high-molecular-weight polyethylene (eUHMWPE), expanded polylactic acid (ePLA), and expanded polyethylene (ePE).

In some embodiments, the non-mammalian biointerface comprises an expanded polymer. In some embodiments, the expanded polymer forms the microstructure of the non-mammalian biointerface. In some embodiments, the expanded polymer is expanded polyurethane (ePU).

In some embodiments, the expanded polymer includes the nutrient phase. This may be achieved by co-blending the nutrient phase with the fluoropolymer resin prior to expansion of the polymer.

In some embodiments, the non-mammalian biointerface comprises a polymer formed by expanded chemical vapor deposition (CVD). In some embodiments, the polymer formed by expanded CVD forms the microstructure of the non-mammalian biointerface. In some embodiments, the polymer formed by expanded CVD is polyparaxylylene (ePPX).

In some embodiments, the non-mammalian biointerfaces described herein can be used to culture non-mammalian cells. Non-mammalian cells are contacted for a sufficient time and under predetermined conditions with a non-mammalian biointerface having desired properties for retaining and viably maintaining the non-mammalian cells until at least some of the non-mammalian cells are retained within the microstructure of the non-mammalian biointerface. In some embodiments, upon retention of the non-mammalian cells by the non-mammalian biointerface, the non-mammalian biointerface can be incubated in a medium conducive to the proliferation of the non-mammalian cells. In other embodiments, the non-mammalian biointerface itself provides a microenvironment conducive to the proliferation of the non-mammalian cells, at least for a period of time (e.g., during temporary transport).

In some embodiments, the non-mammalian biointerfaces described herein can be used as a growth substrate for multicellular non-mammalian organisms. For example, the non-mammalian biointerfaces can be used to support growth of seaweed from spore to mature seaweed. In some embodiments, the non-mammalian cell or group of cells that is to mature into the multicellular non-mammalian organism is contacted for a sufficient time and under predetermined conditions with a non-mammalian biointerface having desired properties for retaining and viably maintaining the non-mammalian cells and supporting growth of a multicellular organism therefrom, until at least some of the non-mammalian cells are retained within the microstructure of the non-mammalian biointerface.

### Use of Non-Mammalian Biointerfaces for Seaweed Cultivation

In certain embodiments, the non-mammalian biointerfaces described herein can be used as an improved growth substrate for the growth and cultivation of seaweed forms (e.g., spores, gametophytes, sporophytes), resulting in improved yield and throughput relative to current cultivation practices.

The current process to cultivate seaweed from spores involves using textured nylon “culture strings” or “seed strings” to which the spores weakly attach during a lab-based seeding process and are then nourished through external nutrient systems. The culture string containing weakly attached juvenile seaweed (gametophytes and sporophytes) is then wound onto ropes at a seaweed farm. Due to the ease in which the seaweed can be damaged, the process is inherently variable in terms of yield and throughput due in large part to the ease in which the seaweed can be damaged from, for example, currents, changes in temperature, and nutrient availability. There exists a need to produce seaweed more efficiently through a more robust process of cultivation primarily through improved stability of juvenile seaweed forms during/after the initial spore seeding and more effective and more specific nutrient delivery systems.

Current approaches to improving stability of juvenile seaweed on culture strings is focused on the surface texture of existing fibers. Indeed, fiber texture of culture strings is very important to the success of seaweed cultivation. However, improvements to surface texture are limited.

In certain embodiments, microstructures resulting from the microporous nature of PTFE fibers and membranes have inter-fibril distances sufficient to retain a wide range of seaweed spore sizes (e.g., 1-200 microns in diameter) that provide a more effective stabilization scaffold plus a unique and very efficient nutrient delivery system from within the microstructure.

Various seaweed spores can be retained by non-mammalian biointerfaces as described herein. Dulse spores are retained by the non-mammalian biointerfaces, and juvenile seaweed growth therefrom with the non-mammalian biointerface providing a growth substrate (see, e.g., FIGS. 7A-70, 9, 10). Nori, kelp, and dulse spores, as well as spores of other seaweed species, or a combinations of different seaweed spore type, can be retained by the non-mammalian biointerfaces. Nori and kelp spores each have a diameter of about 10 μm, while dulse spores have a diameter of about 30 μm. The average inter-fibril distance of fibrillated ePTFE is set to a distance sufficient to allow at least a portion of a seaweed spore to enter into the inter-fibril space and be retained there.

The spores are introduced into the microstructure of the non-mammalian biointerface in a laboratory setting, and gametophytes and sporophytes allowed to mature in a in a manner similar to traditional culture string. Alternatively, the spores can be introduced to the microstructure of the non-mammalian biointerface in the field (i.e., at a seaweed farm site). This in-the-field approach is made possible by the retention properties of the microstructure of the non-mammalian biointerface.

By depositing a material having the presently described microstructure (either with or without spores retained therein) on a rope or cable in the field, the traditional step of wrapping a culture string around a rope line can be skipped. This can be accomplished where the microstructure is provided by a plurality of particles in a dispersion.

In other embodiments, seaweed sporophytes and/or gametophytes are directly introduced into the microstructure of the cultivation substrate. Such direct seeding can reduce the laboratory time required to produce a culture string relative to spore seeding.

Culture strings are traditionally maintained and cultured in a laboratory environment using sterilized sea water. The present non-mammalian biointerfaces, through inclusion of sufficient salt within the microstructure, circumvents the need for the expensive and cumbersome systems required for circulation of sterilized sea water by providing a saline microenvironment within the microstructure. By including a nutrient phase within the microstructure that is sufficient to support seaweed growth, it is possible to avoid having to provide external nutrients to the growing seaweed.

Culture strings must be carefully transported in sea water while avoiding jostling to prevent gametophyte and sporophyte detachment from the string. Conversely, the presently described non-mammalian biointerfaces allow for the gametophytes and sporophytes to be safely transported without sea water. This is achievable by the inclusion of salt and a liquid containing phase within the microstructure, which provides a saline microenvironment having sufficient moisture to support the juvenile seaweed during transport. Furthermore, as the juvenile seaweed is able to grow into the microstructure rather than simply attach superficially to a surface of, e.g., a culture string, loss by detachment is minimized. This beneficial effect extends to the seaweed farm, where currents may detach weakly secured juvenile seaweed.

### EXAMPLES

### Example 1—Porous Polyethylene

Dulse and kelp cultivation trials were conducted on 2 porous polyethylene-based membranes.

Membrane 1 is a gel processed polyethylene membrane measuring 500 millimeters wide, 30 microns thick, with an area density of 18.1 g/m² and an approximate porosity of 36%. This tape was subsequently stretched in the machine direction through a hot air dryer set to 120 degrees Celsius at a stretch ratio of 2:1 with a stretch rate of 4.3%/second. This was followed by a transverse direction stretch in an oven at 130 degrees Celsius at a ratio of 4.7:1 with a stretch rate of 15.6%/second. The resulting membrane possessed the following properties: width of 697 millimeters, thickness of 14 microns, porosity of 66%, and maximum load of 7.65 Newtons×6.23 Newtons and elongation at maximum load of 25.6%×34.3% in the machine direction and transverse directions respectively as tested according to ASTM D412. The membrane had a Gurley Time of 15.7 seconds. Gurley Time is defined as the number of seconds required for 100 cubic centimeters (1 deciliter) of air to pass through 1.0 square inch of a given material at a pressure differential of 4.88 inches of water (0.176 psi) (ISO 5636-5:2003).

Membrane 2 is a commercially available porous polyethylene from Saint Gobain rated as a UE 1 micron lab filter disc. The microstructure of membrane 2 is depicted in FIG. 13.

Membrane samples were secured to 2 inch diameter PVC cups. All samples were sprayed with alcohol and rinsed with freshwater just prior to seeding. Seeding was accomplished by pouring spore solution over samples and allowing spores to settle onto substrate surfaces. Samples were seeded in 10 gallon tanks, and seawater was changed every week. Dulse samples were moved to a 40 gallon fiberglass tank after week 2. Kelp were cultured in 10 gallon tanks. All cultures received aeration. Samples were photographed 2 months after seeding when plants were visible.

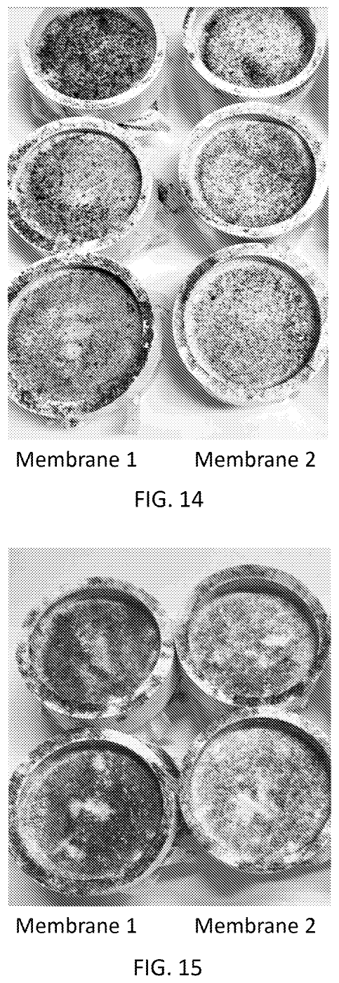

All dulse samples were gently rinsed with freshwater and then dipped into seawater before the evaluation to remove any fouling. Both membrane 1 and 2 showed healthy, medium to high density growth of Dulse seedlings (see FIG. 14). Membrane 1 showed higher density plant growth than Membrane 2. Both Membrane 1 and 2 showed strong seedling attachment and stability.

Kelp samples were lightly rinsed with seawater before photographing. Both membrane 1 and 2 showed healthy, medium to high density growth of Kelp seedlings (see FIG. 15). Membrane 1 showed higher density plant growth than Membrane 2. Both Membrane 1 and 2 showed strong seedling attachment and stability.

### Example 2—Patterned Membranes

A patterned fluoropolymer-based membrane in accordance with certain embodiments was generated with large square areas of low and high porosity. The pattern was in the form of a “checkerboard” design.

Membrane samples were secured to 2 inch diameter PVC cups. All samples were sprayed with alcohol and rinsed with freshwater just prior to seeding. Seeding was accomplished by pouring spore solution over samples and allowing spores to settle onto substrate surfaces. Samples were seeded in 10 gallon tanks, and seawater was changed every week. Dulse samples were moved to a 40 gallon fiberglass tank after week 2. Kelp samples were cultured in 10 gallon tanks. All cultures received aeration. Samples were photographed 2 months after seeding when plants were visible.

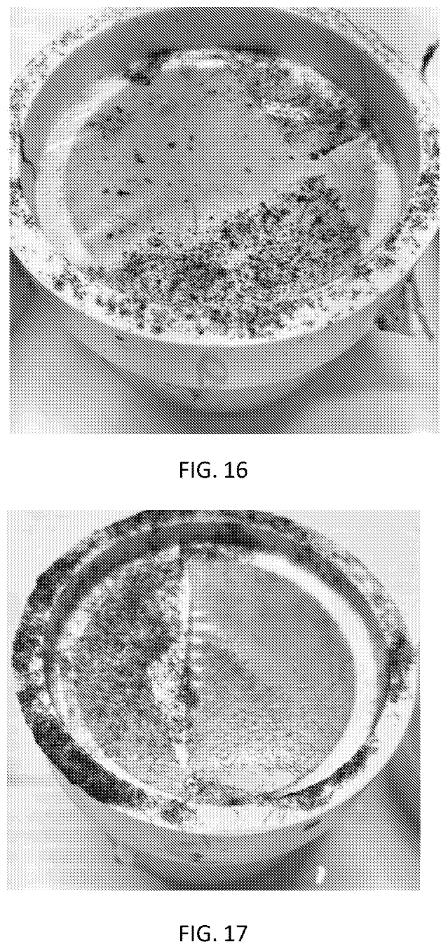

All dulse samples were gently rinsed with freshwater and then dipped into seawater before the evaluation to remove any fouling. With reference to FIG. 16, the checkerboard pattern showed large differences in plant density, with the high porosity (white) squares supporting a healthy, high density covering of plants with strong attachment and the low porosity (clear) squares showing a very low density covering of plants.

Kelp samples were lightly rinsed with seawater before photographing. With reference to FIG. 17, the checkerboard pattern showed large differences in plant density, with the high porosity (white) squares supporting a healthy, high density covering of plants with strong attachment and the low porosity (clear) squares showing a very low density covering of plants.

### Example 3—Direct Sporophyte Seeding

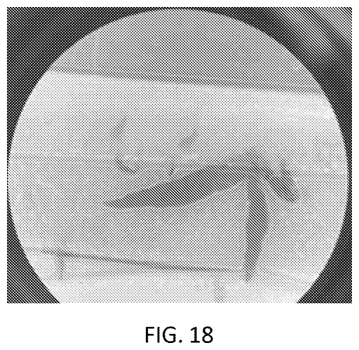

Juvenile sugar kelp sporophytes previously in induction conditions were seeded without any binder onto an experimental membrane of the present disclosure having a width of 4 mm, and a braided polyester control having a diameter of 2 mm. Attachment of the juvenile sporophytes was evaluated for 19 days after seeding. The sporophytes demonstrated attachment and growth on both substrates. Healthy sporophyte growth on the membrane of the present disclosure is depicted in FIG. 18.

To quantify the attachment strength to the two substrates, scores on a scale of 1 to 5 were given to 20 or more sporophytes attached to each substrate, with 1 being very weak attachment and 5 being very strong attachment. The majority of sporophytes attached to the braided polyester control were rated ‘1’, with very weak attachment. The majority of sporophytes attached to the experimental membrane were rated ‘5’, with very strong attachment. The difference in attachment strength between the two substrates was further demonstrated by the ability of sporophytes attached to the experimental membrane to be handed and moved with tweezers while remaining attached to the substrate. The sporophytes attached to the braided polyester control could not be handled, moved, or even agitated without being detached from the substrate.

### Example 4—Mushroom Cultivation

Peat moss is typically used as a casing material in mushroom cultivation on top of a compost layer to support the change of mycelium from vegetative growth (in the compost) to reproductive growth (in the casing layer) and subsequent fruiting of mushrooms. In mushroom cultivation, the first harvest (or flush) of mushrooms is of the highest quality in terms of appearance, consistency and value. After harvesting, the second and subsequent flushes continually decline in quality and value. After 3 or 4 flushes, the peat moss and compost are removed and replaced to start a new cultivation cycle.

A fabric weave of highly porous fibers of the present disclosure was placed approximately centrally within the typical peat moss casing layer, which had a thickness of about 2 inches. The standard cultivation cycle for white button mushrooms (Agaricus bisporus) was carried out. The first flush of mushrooms were of very high quality in terms of appearance and consistency. After harvesting, the cultivation substrate was inspected and was found to have been extensively colonized by the mycelium during the conversion from vegetative to reproductive growth. In addition, the mycelium network from the cultivation substrate extended significantly in the peat moss phase above and below the substrate.

After the first flush was harvested, the substrate and the 1 inch peat moss covering the substrate was removed to reveal the remaining 1 inch of peat moss which also showed extensive colonization of mycelium. The cultivation cycle was repeated and the mycelium became reproductive producing mushroom fruit for the second harvest (flush). After the substrate is removed, the quality (appearance and consistency) of the mushroom after the second flush was comparable to the first flush and regarded as very high quality.

Without wishing to be bound by any particular theory, it appears that the biointerface substrate supports healthy mycelium development and colonization, while providing some level of protection to underlying peat moss from pathogens and contaminants.

The substrate can be reused, and the significantly higher quality of the second flush justifies the initial cost of the substrate.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A non-mammalian biointerface comprising a microstructure configured to retain and viably maintain viruses or non-mammalian cells, the microstructure being characterized by an average inter-fibril distance up to and including 200 μm.

2. A non-mammalian biointerface comprising a microstructure configured to retain and viably maintain viruses or non-mammalian cells, the microstructure configured to retain viruses or non-mammalian cells at least partially within the microstructure, the microstructure being characterized by an average pore size of up to and including 200 μm.

3. The non-mammalian biointerface of claim 1, wherein the microstructure is characterized by an average inter-fibril distance from 1 to 200 μm.

4. The non-mammalian biointerface of claim 2 or claim 3, wherein the microstructure is characterized by an average pore size from 1 to 200 μm.

5. The non-mammalian biointerface of any one of claims 1-4, wherein the microstructure is configured to retain spores.

6. The non-mammalian biointerface of any one of claims 1-4, wherein the microstructure is configured to retain bacteria.

7. The non-mammalian biointerface of any one of claims 1-4, wherein the microstructure is configured to retain microbes.

8. The non-mammalian biointerface of any one of claims 1-7, further comprising a nutrient phase associated with at least a portion of the non-mammalian biointerface.

9. The non-mammalian biointerface of claim 8, wherein at least a portion of the nutrient phase is located within the microstructure, located on the microstructure, or located both within the microstructure and on the microstructure.

10. The non-mammalian biointerface of claim 8 or claim 9, wherein the nutrient phase is present as a coating on a surface of the non-mammalian biointerface.

11. The non-mammalian biointerface of any one of claims 8-10, wherein the nutrient phase acts as a chemoattractant to selectively attract the viruses or non-mammalian cells to predetermined locations of the non-mammalian biointerface to which the nutrient phase is applied or included.

12. The non-mammalian biointerface of any of claims 8-11, wherein the nutrient phase is configured to i) promote growth and/or proliferation of the viruses or non-mammalian cells within the microstructure, and/or ii) maintain and/or encourage attachment to and integration within the microstructure of the viruses or non-mammalian cells to the microstructure.

13. The non-mammalian biointerface of any one of claims 1-12, further comprising a liquid containing phase associated with at least a portion of the non-mammalian biointerface.

14. The non-mammalian biointerface of claim 13, wherein at least a portion of the liquid containing phase is entrained within the microstructure, entrained on the microstructure, or entrained both within the microstructure and on the microstructure.

15. The non-mammalian biointerface of claim 13 or claim 14, wherein the liquid containing phase is present as a coating on a surface of the non-mammalian biointerface.

16. The non-mammalian biointerface of any one of claims 13-15, wherein the liquid containing phase comprises a hydrogel, a slurry, a paste, or a combination thereof.

17. The non-mammalian biointerface of any one of claims 1-16, further comprising a plurality of viruses or non-mammalian cells retained by the microstructure of the non-mammalian biointerface.

18. The non-mammalian biointerface of any one of claims 1-17, wherein the non-mammalian biointerface includes a fibrillated material having a microstructure including a plurality of fibrils defining an average inter-fibril distance.

19. The non-mammalian biointerface of any one of claims 1-18, wherein the non-mammalian biointerface comprises a material having an average density from 0.1 to 1.0 g/cm³.

20. The non-mammalian biointerface of claim 19, wherein the non-mammalian biointerface includes a growth medium comprising the material, and a ratio of the average inter-fibril distance (μm) to the average density (g/cm³) of the fibrillated material is from 1 to 2000.

21. The non-mammalian biointerface of any one of claims 1-20, wherein the non-mammalian biointerface is configured as a fiber, a membrane, a woven article, a non-woven article, a braided article, a knit article, a fabric, a particulate dispersion, or combinations of two or more of the foregoing.

22. The non-mammalian biointerface of any one of claims 1-21, wherein the non-mammalian biointerface includes at least one of a backer layer, a carrier layer, a laminate of a plurality of layers, a composite material, or combinations thereof.

23. The non-mammalian biointerface of any one of claims 1-22, wherein at least a portion of the non-mammalian biointerface is hydrophilic.

24. The non-mammalian biointerface of any one of claims 1-23, wherein at least a portion of the non-mammalian biointerface is hydrophobic.

25. The non-mammalian biointerface of any one of claims 1-24, wherein one or more portions of the non-mammalian biointerface is hydrophobic and one or more portions of the non-mammalian biointerface is hydrophilic such that the non-mammalian biointerface is configured to selectively encourage retention of the viruses or non-mammalian cells in the one or more hydrophilic portions of the non-mammalian biointerface.

26. The non-mammalian biointerface of any one of claims 1-25, wherein the non-mammalian biointerface comprises an expanded fluoropolymer.

27. The non-mammalian biointerface of any one of claims 8-25, wherein the biointerface comprises an expanded fluoropolymer wherein the nutrient phase is co-blended with the expanded fluoropolymer.

28. The non-mammalian biointerface of claim 26 or claim 27, wherein the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), and expanded polytetrafluoroethylene (ePTFE).

29. The non-mammalian biointerface of any one of claims 1-25, wherein the non-mammalian biointerface comprises an expanded thermoplastic polymer.

30. The non-mammalian biointerface of claim 29, wherein the expanded thermoplastic polymer is one of: expanded polyester sulfone (ePES), expanded ultra-high-molecular-weight polyethylene (eUHMWPE), expanded polylactic acid (ePLA), and expanded polyethylene (ePE).

31. The non-mammalian biointerface of any one of claims 1-25, wherein the non-mammalian biointerface comprises an expanded polymer.

32. The non-mammalian biointerface of any one of claims 8-25 and 31, wherein the non-mammalian biointerface comprises an expanded polymer wherein the nutrient phase is co-blended with the expanded polymer.

33. The non-mammalian biointerface of claim 31 or claim 32, wherein the expanded polymer is expanded polyurethane (ePU).

34. The non-mammalian biointerface of any one of claims 1-25, wherein the non-mammalian biointerface comprises a polymer formed by expanded chemical vapor deposition (CVD).

35. The non-mammalian biointerface of claim 34, wherein the polymer formed by expanded CVD is expanded polyparaxylylene (ePPX).

36. The non-mammalian biointerface of any one of claims 1-35, further comprising a bioactive agent associated with the non-mammalian biointerface.

37. The non-mammalian biointerface of any one of claims 1-36, further comprising an adhesive applied to a surface of the microstructure, imbibed within the microstructure of the non-mammalian biointerface, or both applied to a surface of the microstructure and imbibed within the microstructure of the non-mammalian biointerface.

38. The non-mammalian biointerface of any one of claims 1-36, further comprising a salt associated with the microstructure of the non-mammalian biointerface.

39. The non-mammalian biointerface of claim 38, wherein the salt is sodium chloride (NaCl).

40. The non-mammalian biointerface of any one of claims 1-39, wherein the microstructure includes a pattern of higher density portions and lower density portions, the lower density portions corresponding to a portion of the microstructure configured to retain spores on and/or within the microstructure of the microstructure.

41. The non-mammalian biointerface of claim 40, wherein the lower density areas are characterized by a density of 1 g/cm³ or less and the higher density portions are characterized by a density of 1.7 g/cm³ or more.

42. The non-mammalian biointerface of any one of claims 1-41, wherein the microstructure includes a pattern of higher porosity portions and lower porosity portions, the lower porosity portions corresponding to a portion of the microstructure configured to retain viruses or non-mammalian cells within the microstructure of the non-mammalian biointerface.

43. The non-mammalian biointerface of any one of claims 1-41, wherein the microstructure includes a pattern of higher porosity portions and lower porosity portions, the higher porosity portions corresponding to a portion of the microstructure configured to retain viruses or non-mammalian cells within the microstructure of the non-mammalian biointerface.

44. The non-mammalian biointerface of any one of claims 1-43, wherein the microstructure includes a pattern of greater inter-fibril distance portions and lower inter-fibril distance portions, the lower inter-fibril distance portions corresponding to the portion of the microstructure configured to retain spores within the microstructure of the non-mammalian biointerface.

45. The non-mammalian biointerface of any one of claims 1-43, wherein the microstructure includes a pattern of greater inter-fibril distance portions and lower inter-fibril distance portions, the greater inter-fibril distance portions corresponding to the portion of the microstructure configured to retain spores within the microstructure of the non-mammalian biointerface.

46. The non-mammalian biointerface of claim 44 or claim 45, wherein the pattern is an organized or selective pattern.

47. The non-mammalian biointerface of claim 44 or claim 45, wherein the pattern is a random pattern.

48. The non-mammalian biointerface of any one of claims 1-25 and 36-47, wherein the microstructure is provided by a plurality of particles in a dispersion formulated for deposition onto a backer layer or a carrier substrate to form the non-mammalian biointerface.

49. The non-mammalian biointerface of claim 48, wherein the plurality of particles comprises particles of an expanded fluoropolymer.

50. The non-mammalian biointerface of claim 49, wherein the expanded fluoropolymer is one of: expanded fluorinated ethylene propylene (eFEP), porous perfluoroalkoxy alkane (PFA), expanded ethylene tetrafluoroethylene (eETFE), expanded vinylidene fluoride co-tetrafluoroethylene or trifluoroethylene polymer (eVDF-co-(TFE or TrFE)), and expanded polytetrafluoroethylene (ePTFE).

51. The non-mammalian biointerface of claim 48, wherein the plurality of particles comprises particles of an expanded thermoplastic polymer.

52. The non-mammalian biointerface of claim 51, wherein the expanded thermoplastic polymer is one of: expanded polyester sulfone (ePES), expanded ultra-high-molecular-weight polyethylene (eUHMWPE), expanded polylactic acid (ePLA), and expanded polyethylene (ePE).

53. The non-mammalian biointerface of claim 48, wherein the plurality of particles comprises particles of an expanded polymer.

54. The non-mammalian biointerface of claim 53, wherein the expanded polymer is expanded polyurethane (ePU).

55. The non-mammalian biointerface of claim 48, wherein the plurality of particles comprises a polymer formed by expanded chemical vapor deposition (CVD).

56. The non-mammalian biointerface of claim 55, wherein the polymer is polyparaxylylene (ePPX).

57. A method for cultivating a non-mammalian cell, comprising contacting a population of non-mammalian cells with the non-mammalian biointerface of any one of claims 1-56 until at least a portion of the population of non-mammalian cells is retained by the non-mammalian biointerface.
